(19) United States
(12) Patent Application Publication
(10) Pub. No.: US 2023/0338200 A1
(43) Pub. Date: Oct. 26, 2023

(54) CATAMENIAL PROTECTION KIT

(71) Applicant: Moons, Inc., Newburgh, NY (US)

(72) Inventor: Rachel GELLER, Newburgh, NY (US)

(21) Appl. No.: 18/135,973

(22) Filed: Apr. 18, 2023

Related U.S. Application Data
(60) Provisional application No. 63/332,133, filed on Apr. 18, 2022.

(51) Int. Cl. A61F 13/20; A61F 13/26

(52) U.S. Cl. CPC A61F 13/202; A61F 13/266

(57) ABSTRACT

A catamenial protection kit includes a tampon device having an applicator configured to house and eject a tampon plug for use. The tampon plug has a diameter in a range between ⅜ and ⅝ inches and is removably wrapped in a hygienic wrapping. The kit further includes an absorbent device which is adapted to be releasably affixed to an undergarment of a user. The absorbent device includes an absorbent zone, a non-absorbent peripheral zone circumscribing at least a portion of the absorbent zone, an adhesive provided on a surface opposite of the absorbent zone, and a foldable hygienic wrapper for retaining the absorbent device. The wrapped tampon device is emplaced within the foldable hygienic wrapper. A flexible outer wrapper is configured and dimensions to retain and seal the folded wrapper about the tampon device to form the catamenial protection kit.

### CROSS REFERENCE TO RELATED APPLICATIONS

This patent application claims the benefit of U.S. Provisional Application No. 63/332,133, filed Apr. 18, 2022, the content of which is incorporated by reference herein in its entirety.

### FIELD OF INVENTION

The present invention relates to a three-piece catamenial protection kit which includes the combined use of an applicator with a tampon and an ultra-thin liner to permit an improved solution to a user's comfort during the sloughing off period of her menstrual cycle.

### BACKGROUND OF RELATED PRIOR ART

Over the years there have been many catamenial products developed to address the capture of sloughed off menses. In the main, they have taken the form of either an absorbent pad or a vaginally inserted tampon.

As better absorbent materials were developed, the technology was applied to tampons, both to increase the level of absorbency and address issues of what has been termed “by-pass” leakage in which the tampon material does not fully conform to the uterine walls and despite available absorbency nonetheless the menses stains undergarments. This in turn, in the 1970/1980, led to what was termed in the industry as the absorbency wars in which the makers of tampons competed to develop maximum absorbency and widthwise expansion for their respective products.

However, with the added absorbency and contact came a medical condition known as toxic shock syndrome. This in turn led to FDA regulations relating to absorbency levels and the creation of standards and an end to a cycle of absorbency level competition.

The key marketers of tampons maintained the absorbency at or near permissible levels and sought improvements in delivery devices such as applicators.

In general, however, the market for catamenial devices has remained divided between insertable devices, i.e., tampons and absorbent pads with permissible, but maximized “super” sized tampons available for times of heavier menstrual flow.

As well, it has been suggested that a fully sized pad be used in conjunction with a traditionally sized tampon to capture fluid leakage not absorbed by the tampon.

Therefore, a women's choice, if a tampon user for the 3-6 days of menstruation, was either a relatively larger tampon alone or with a bulky supplemental pad.

### SUMMARY

The present invention provides a more comfortable alternative, namely through the use of a smaller dimensioned high-performing capsule tampon and a companion slim super absorbent liner which are available together to provide a system for enhanced comfort and convenience.

Preferably, these companion devices can be packaged together to form a catamenial protection kit which includes a storage packet having a size comparable to that of a standard tampon packet, thereby providing a convenient packet that can easily be fit into a purse for ready access to both the capsule tampon and the absorbent liner. Advantageously, the packet can also serve as a disposable container for the soiled products versus flushing the waste, which is generally prohibited due to the adverse effects on the environment and plumbing.

In one embodiment, a catamenial protection kit comprises: a tampon device including an applicator configured to house and eject a tampon plug for use, said tampon plug having a diameter in a range between ⅜ and ⅝ inches, said tampon plug removably wrapped in a hygienic wrapping; an absorbent device adapted to be releasably affixed to an undergarment of a user, said absorbent device including: an absorbent zone, a non-absorbent peripheral zone circumscribing at least a portion of the absorbent zone, an adhesive provided on a surface opposite of the absorbent zone, a foldable hygienic wrapper for retaining said absorbent device, and wherein the wrapped tampon device is emplaced within said foldable hygienic wrapper; and a flexible outer wrapper configured and dimensions to retain and seal the folded wrapper about the tampon device to form said catamenial protection kit.

In one aspect of the catamenial protection kit, the foldable hygienic wrapper circumscribes the wrapped tampon device. In another aspect, the flexible outer wrapper is generally rectilinear in shape. In yet another aspect, the absorbent device has opposing distal ends that are tapered and rounded in shape. In still another aspect, the foldable wrapper of the absorbent device is removably attached to the opposite surface of the absorbent zone by said adhesive. In another aspect, the tampon plug is cylindrical in shape with a longitudinal length of approximately 1¾ inches and a diameter of approximately ½ inch.

### BRIEF DESCRIPTION OF THE DRAWINGS

FIG. 1 is a front view of a sealed tampon packet containing a catamenial protection kit in accordance with the present invention;

FIG. 2 is an isometric view of the tampon packet of FIG. 1 in an unsealed state depicting its contents including a wrapped tampon applicator and wrapped liner packet folded around the wrapped applicator;

FIG. 3 is an expanded view of the wrapped applicator and liner packet of FIG. 2;

FIG. 4 is an isometric view of the applicator after being unwrapped from its wrapper;

FIG. 5 is an isometric view of the applicator of FIG. 4;

FIG. 6 is an isometric view of the applicator of FIG. 5 depicting a plunger in an extended position;

FIG. 7 is a perspective end view of a tip the applicator of FIG. 4 depicting a plurality of flexible members for releasing a tampon;

FIG. 8 is a perspective end view of a tip the applicator of FIG. 4 depicting the tampon being released from the applicator;

FIG. 9 is a plan view of the liner packet in an unfolded state in accordance with the present invention;

FIG. 10 is a plan view of the liner of FIG. 9; and

FIG. 11 is a plan view of the liner of FIG. 9 depicting an adhesive backing in accordance with the present invention.

To facilitate understanding of the invention, identical reference numerals have been used, when appropriate, to designate the same or similar elements that are common to the figures.

### DETAILED DESCRIPTION

The present invention, provides a more comfortable alternative, namely through the use of a smaller dimensioned high-performing capsule tampon and a companion slim super absorbent liner, which are available together to provide a system for enhanced comfort and convenience. Preferably, these companion devices are packaged together in a storage packet having a size comparable to that of a standard tampon packet, thereby providing a convenient packet that can easily be fit into a purse or pocket for ready access to both the capsule tampon and the ultra-thin absorbent liner.

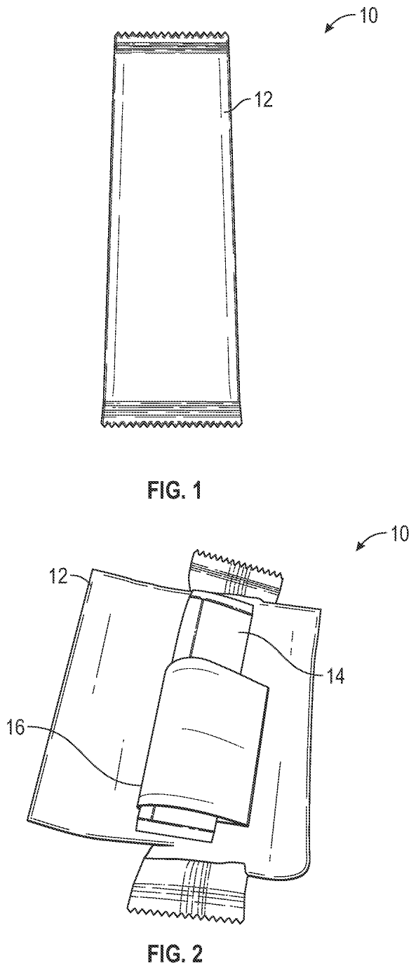

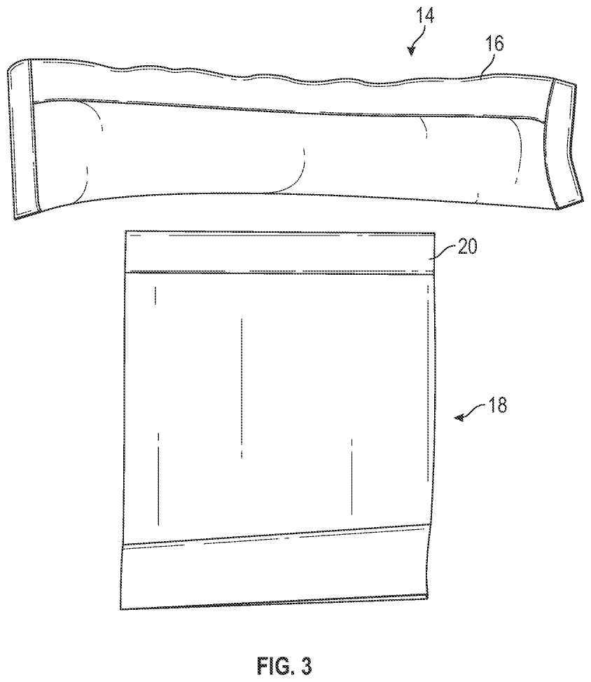

Referring to FIGS. 1-3, a tampon packet 10 is illustratively shown sealed by a water-resistant wrapper 12, as illustratively shown in FIG. 1. The tampon packet 10 includes an applicator packet 14 and a liner packet 18, as illustratively shown in FIGS. 2 and 3. The applicator packet 12 is preferably sealed by a sanitary water-resistant wrapper 14. Similarly, the liner packet is preferably sealed by a sanitary water-resistant wrapper 16.

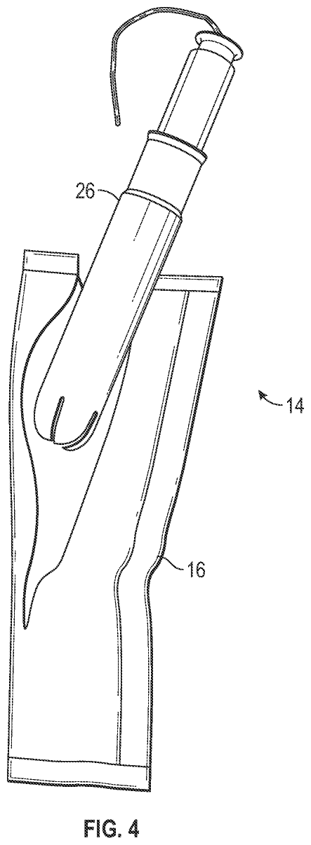

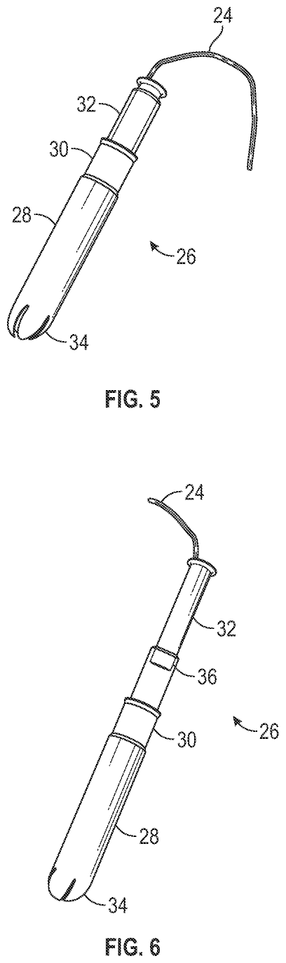

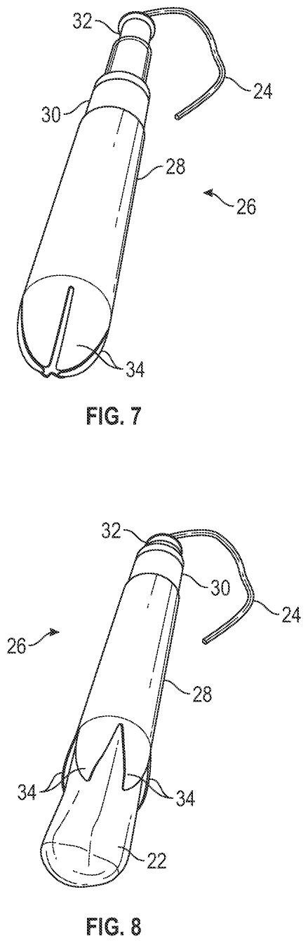

Referring to FIGS. 4-8, the tampon 22 is preferably stored in an applicator 26 which readily permits insertion of the tampon 22 into the vaginal canal. Preferably, the tampons 22 are provided in the standard two absorbency ranges, i.e., regular with a 6-9 grams absorbency range and super with a 9-12 grams absorbency range. Although there is no specific dimensional size for tampons as they vary and are identified by absorbency ranges, the tampon 22 of the present invention is generally smaller and thinner than more conventional tampons and is generally of cylindrical shape with a longitudinal length of approximately 1¾ inches and a diameter in a range of approximately ⅜ to ⅝ inches with a removal string 24 affixed to the base. A string 24 is attached to a rear end of the tampon 22 and extends a sufficient length therefrom to enable manual removal of the tampon 26 from the vaginal cavity in a well-known manner.

The applicator 26 includes an elongated, tubular barrel 28 having a proximal end within which the tampon 22 retained, and a finger grip 30 positioned at a distal end of the barrel 28. The applicator 26 is preferably cylindrical in shape, although such shape is not considered limiting. A slidable, tubular plunger 32 is reciprocally mounted in the distal end of the barrel 28 and is configured to eject the tampon 22 from the barrel 28. The plunger 32 can include a shoulder or flange 36 (FIG. 6) which acts as a stop to prevent removal of the plunger 32 from the distal end of the barrel 28. The barrel is sized to accommodate the entire tampon 22, while the string 24 is routed through and extends a predetermined length from the tubular plunger 26. Preferably, the proximal end, i.e., the barrel tip 15 is formed by a plurality of flexible segmented members 34 which are configured to flex and open while the tampon 22 is being ejected from the barrel 28, as shown in FIG. 8. In the embodiment shown in FIGS. 4-8, five flexible members 34 are illustratively provided, although such quantity is not considered limiting. Alternatively, the applicator can include a single opening without the flexible members 34. During use, the user grasps the finger grip 30 to insert the barrel 28 into the vaginal cavity and presses the plunger 32, which forces the tampon 22 out of the barrel 28 through the flexible segmented members 34 and into the vaginal cavity. The applicator 26 is then removed from the vaginal cavity and appropriately discarded.

The applicator is fabricated from medical-grade plastic material, such as polyethylene, and/or other well-known medical-grade materials. Alternatively, the applicator can be fabricated from a bio-degradable material, such as cardboard in a well-known manner. A person of ordinary skill in the art will appreciate that the type of applicator and the configuration of its plunger and opening for dispensing of the tampon 22 is not considered limiting.

It should be understood that although the tampon 22 has absorbency potential with ordinary norms, its smaller size, while promoting ease of insertion and comfort, satisfies the FDA standards of absorbency as any other market-leading regular and super tampon.

Moreover, even if leakage occurs due to less than the full use of absorbency potential of the tampon 22 is not encountered, as is the case with tampons in general, a woman needs to consider when it is appropriate to replace the tampon. Generally, a light tug on the string 24 is suggested and if there is a sensation that the tampon 22 starts to pull out easily, it is best to remove it. However, a more telling and somewhat upsetting time is when the staining of undergarments is observed.

For that reason, particularly during times of heavier menstruation, some women also wear a standard absorbent pad. However, this practice for many is contrary to the very reason a woman has selected the use of a tampon instead of the use of a pad.

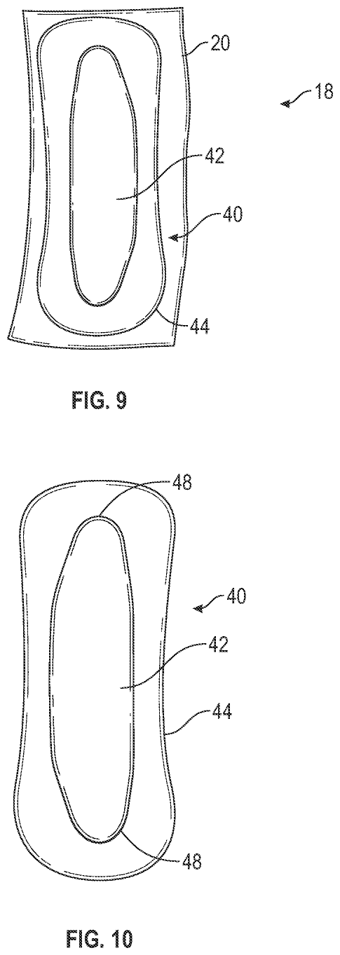

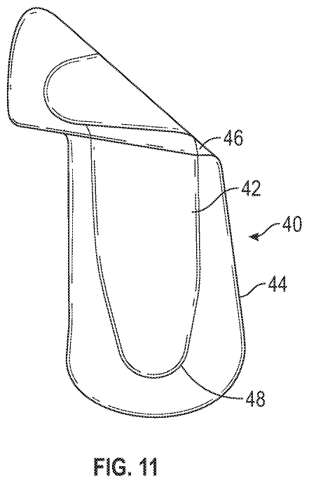

To address this issue and referring to FIGS. 9-11, the present invention provides a small flexible absorbent liner 40, with an absorbent face 42 secured to a larger substrate 44 with an adhesive backing 46 on the substrate 44 which is releasably secured to the interior surface of the liner wrapper, i.e., packaging material 20, such that the packaging material acts to not only contain the liner but, when removed, exposes the adhesive backing 46 of the substrate 44.

The liner 20 in its preferred embodiment includes an elongated oval or gunboat shaped absorbent surface 42 approximately 5 inches in length, 1¼ inches at its widest section with tapered and rounded distal ends 48. The absorbent surface is mounted on the larger substrate 44 so that when released from packaging material surface on which it is mounted a larger surface area is provided for removably securing the liner to an undergarment and aligns the absorbent layer along the vaginal opening and thus be in a position to capture any leakage. As a consequence, a visual indicator of leakage is provided without the need for a bulky pad.

Referring now to FIGS. 2-3, the liner 40 due to its slim size can be folded in half or in thirds to reduce its size and permit it to be packaged with the tampon 22 into a single packet 10. In particular, the liner packet 16 can be wrapped around the applicator packet 14, as illustratively shown in FIG. 2, to minimize space and packaging in the tampon packet wrapper 12.

The foregoing specific embodiments represent just some of the ways of practicing the present invention. Many other embodiments are possible within the spirit of the invention. Accordingly, the scope of the invention is not limited to the foregoing specification, but instead is given by the appended claims along with their full range of equivalents.

### REFERENCE NUMBERS

10 tampon packet
12 tampon packet wrapper
14 applicator packet
16 applicator packet wrapper
18 liner packet
20 liner packet wrapper
22 tampon plug
24 tampon string
26 applicator
28 barrel
30 finger-grip
32 plunger
34 flexible members
36 plunger shoulder
40 absorbent liner
42 absorbent face
44 substrate
46 adhesive backing
48 distal ends of liner

What is claimed is:

1. A catamenial protection kit comprising:
a tampon device including an applicator configured to house and eject a tampon plug for use, said tampon plug having a diameter in a range between ⅜ and ⅝ inches, said tampon plug removably wrapped in a hygienic wrapping;
an absorbent device adapted to be releasably affixed to an undergarment of a user, said absorbent device including:
an absorbent zone,
a non-absorbent peripheral zone circumscribing at least a portion of the absorbent zone,
an adhesive provided on a surface opposite of the absorbent zone,
a foldable hygienic wrapper for retaining said absorbent device, and
wherein the wrapped tampon device is emplaced within said foldable hygienic wrapper; and
a flexible outer wrapper configured and dimensions to retain and seal the folded wrapper about the tampon device to form said catamenial protection kit.

2. The catamenial protection kit of claim 1, wherein the foldable hygienic wrapper circumscribes the wrapped tampon device.

3. The catamenial protection kit of claim 1, wherein the flexible outer wrapper is generally rectilinear in shape.

4. The catamenial protection kit of claim 1, wherein the absorbent device has opposing distal ends that are tapered and rounded in shape.

5. The catamenial protection kit of claim 1, wherein the foldable wrapper of the absorbent device is removably attached to the opposite surface of the absorbent zone by said adhesive.

6. The catamenial protection kit of claim 1, wherein the tampon plug is cylindrical in shape with a longitudinal length of approximately 1¾ inches and a diameter of approximately ½ inch.
